(19) United States
(12) Patent Application Publication
(10) Pub. No.: US 2024/0092722 A1
(43) Pub. Date: Mar. 21, 2024

(54) BETA-HYDROXYBUTYRATE WITH PURE BIOBASED CARBON CONTENT AND METHODS FOR PRODUCING THE SAME

(71) Applicant: NANJING NUTRABUILDING BIO-TECH CO., LTD., Nanjing (CN)

(72) Inventors: Kylin LIAO, Nanjing (CN); Ronghua YI, Nanjing (CN); Jiasen YANG, Nanjing (CN); Hailong DONG, Nanjing (CN)

(21) Appl. No.: 18/517,037

(22) Filed: Nov. 22, 2023

Related U.S. Application Data
(63) Continuation of application No. PCT/CN2022/094914, filed on May 25, 2022.

(30) Foreign Application Priority Data
May 25, 2021 (WO) PCT/CN2021/095760

(51) Int. Cl. C07C 59/01; C12P 7/42

(52) U.S. Cl. CPC C07C 59/01; C12P 7/42

(57) ABSTRACT

Among others, the present invention provides for beta-hydroxybutyrate (e.g., R—BHB) acids and/or salts with pure biobased carbon content (e.g., 100% as measured by Carbon-14 analysis). The present invention also provides methods for preparing beta-hydroxybutyrate (e.g., R—BHB) acid and salt with pure biobased carbon content, comprising chemical synthesis and/or biosynthesis process. For instance, the method may include biological fermentation, and/or bioenzymatic process.

### CROSS-REFERENCE TO RELATED APPLICATION

This application is a continuation application of international patent Application No. PCT/CN2022/094914, filed on May 25, 2022, which claims the priority of the international application No. PCT/CN2021/095760, filed on May 25, 2021, the contents of which are incorporated herein by reference in their entirety.

### FIELD OF THE DISCLOSURE

The present disclosure generally relates to the field of health food and dietary supplements and, more particularly, relates to beta-hydroxybutyrate with pure biobased carbon content and methods for producing the same.

### BACKGROUND

Beta-hydroxybutyrate, also known as BHB, is one of the three “physiological” ketones that are produced by the body (specifically, in the liver). Ketone bodies may achieve a number of benefits, such as assisting in breakdown of fat, accelerating circulation of blood, activating the formation of new blood cells, improving gene expression, providing powerful energy to brain, bone, myocardial tissues, reducing activity of some inflammatory molecules (TNF-α), etc. Moreover, BHB can improve high-intensity exercise performance, suppress appetite, and alleviate age-related cognitive degeneration. Further, there has been experiment manifested that BHB supplement could extend the lifespan of Caenorhabditis elegans by as much as 20%, similar to calorie restriction. See, e.g., Edwards, C., et al., D-beta-hydroxybutyrate extends lifespan in C. elegans. Aging (Albany NY), 2014. 6(8): p. 621-44.

BHB is known as a chiral molecule with two forms of enantiomer. One form is R—BHB, which is the molecule produced normally in human and animal metabolism. Fasting, exercise, and ketogenic diets can all boost levels of R—BHB in the blood. R—BHB is the only form of BHB that can break down into acetyl-CoA and, eventually, ATP for energy. The other form is S—BHB, which is actually an intermediate, as a step before R—BHB being produced from fatty acid oxidation. In fact, S—BHB doesn't even stay around long enough to leave the body's mitochondria and enter circulation. In a nutshell, R—BHB is the primary product of endogenous ketogenesis that participates in adenosine triphosphate (ATP) production, whereas S—BHB may retain the signaling functions attributed to BHB and has slower metabolism than R—BHB. See, e.g., Newman, J. C. and E. Verdin, Ketone bodies as signaling metabolites. Trends Endocrinol Metab, 2014. 25(1): p. 42-52. Accordingly, R—BHB is known to be superior to S—BHB.

On the other hand, while it is very important to manufacture products with high purity of biobased carbon content in order to protect human body and environment, conventional R—BHB have been typically manufactured from cheap and abundant petrochemicals, coal, and other fossil sources, resulting in the absence of biobased carbon content nowadays. Obviously, these kinds of R—BHB aren't green or environment friendly, and are also averse to human health. Further, other known methods for preparing R—BHB may be complex and difficult at the low yield, or require expensive raw materials.

Radiocarbon isotope (also known as Carbon-14, C14 or 14C) has been well used to accurately and efficiently measure products from biobased sources (versus fossil-derived constituents). Specifically, radiocarbon isotope is a naturally occurring isotope of carbon that is radioactive and decays in such a way that there is none left after about 45,000 years following the death of a plant or animal. Moreover, accelerator mass spectrometer (AMS) is the most common method used for Carbon-14 measurement, as AMS instrumentation is able to detect very low concentrations of atoms of specific elements according to their atomic weight. Such Carbon-14 results can be reported according to international standards, such as International Organization for Standardization (ISO) 16620-2:2015 and the American Society for Testing and Materials (ASTM) D6866, which provide a standard test method to distinguish biomass-based components from fossil fuel-derived sources through the use of Carbon-14 analysis. Accordingly, Carbon-14 results are reported as % biobased (a fraction of biobased carbon to total carbon), ranging from 0% to 100%. While 100% represents a pure natural source, 0% represents no natural source, and a value in between represents a mixture of natural and fossil sources. The higher the percentage of biobased carbon content, the greater the proportion of naturally sourced components exists in the material.

To overcome the drawbacks of conventional BHB, it is therefore desired to provide improved, healthier, safer, and natural BHB (particularly R—BHB), and effective production methods thereof. Importantly, R—BHB can be measured accurately by Carbon-14 analysis to determine that the products have pure (e.g., 100%) biobased carbon content, which comes from plants or animal by-products (biomass) living in the natural environment.

### SUMMARY

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

This invention generally relates to BHB (e.g., R—BHB) acid and/or salts with pure biobased carbon content (especially 100% as measured by Carbon-14 analysis), together with preparation methods thereof. Particularly, according to the present invention, pure biobased carbon content of BHB (e.g., R—BHB) acid and/or salts was identified by Carbon-14 analysis and this BHB (e.g., R—BHB) can be obtained by chemical synthesis and biosynthesis including, e.g., biological fermentation, bio-enzymatic method. Furthermore, the synthetic processes according to the present invention are simple, easy to control and repeat, effective and efficient, environment friendly and at a low cost and high yield. Biobased content is based upon the sources of the raw material, thus the raw materials of prepared BHB (e.g., R—BHB) are natural and environmental-friendly. The prepared R—BHB acid or salts with pure biobased carbon content are natural green, healthier, and safer than the existing R—BHB acid and/or salts in the market. The BHB (e.g., R—BHB) acid and/or salts with pure biobased carbon content according to the present invention may be widely used in dietary supplementary, food additives and/or pharmaceuticals, and are advantageous for human body and environment.

One aspect of the present invention provides a beta-hydroxybutyrate (BHB) acid and/or salt or a composition comprising such a BHB acid and/or salt, wherein the BHB acid/salt comprises biobased carbon content, and the percentage of biobased carbon content is about 100% as measured by Carbon-14 analysis.

In some embodiments, the beta-hydroxybutyrate (BHB) acid further comprises an ethyl alcohol which reacts with the BHB acid to form a BHB ethyl ester.

In some embodiments, the BHB salt comprises a BHB metal salt. For instance, the BHB salt may be formed from sodium, calcium, magnesium, or a mixture thereof.

In some embodiments, the BHB salt comprises R—BHB, RS—BHB, S—BHB form, or a mixture thereof.

In some embodiments, the BHB acid and/or salt has a chemical purity of at least about 90%.

In some embodiments, the BHB acid and/or salt has a chemical purity of at least about 95%, an optical purity of at least about 95%, and/or is formed with a molar yield of at least about 80%. In some further embodiments, the BHB acid and/or salt has a chemical purity of at least about 98%, an optical purity of at least about 98%, and/or is formed with a molar yield of at least about 85%.

In some embodiments, composition is to be administrated by a mammal, for promoting and/or sustaining ketosis in the mammal (e.g., human).

Another aspect of the present invention provides a beta-hydroxybutyrate (BHB) acid or salt comprising about 100% biobased carbon content.

In some embodiments, the BHB acid further comprises an ethyl alcohol which reacts with the BHB acid to form a BHB ethyl ester.

In some embodiments, the BHB salt is formed with a metal ion.

In a further aspect, the present invention relates to a method for preparing beta-hydroxybutyrate (BHB) (e.g., R—BHB) acid and/or salt, comprising a chemical synthesis or biosynthesis process for enabling pure biobased carbon content in the prepared R—BHB acid and/or salts, wherein the chemical synthesis or biosynthesis processes comprises a biological fermentation, and/or a bio-enzymatic method. For instance, the BHB (e.g., R—BHB) according to the present invention may be produced by a biosynthesis process.

In some embodiments, the method comprises steps of (a) construction of recombinant strains; (b) fermentation of the recombinant bacteria; and (c) extraction and purification of BHB acid and/or salt.

In some embodiments, the method further comprises a step of bio-enzyme catalysis after the process of fermentation of the recombinant bacteria.

In some embodiments, the percentage of biobased carbon content in the prepared BHB acid or salt is about 100% as measured by Carbon-14 analysis.

In some embodiments, the prepared BHB acid comprises a BHB ethyl ester.

In some embodiments, the prepared BHB salt comprises a BHB metal salt. Examples of the metal forms comprises sodium, calcium, magnesium, or a mixture thereof.

In some embodiments, the prepared BHB salt comprises R—BHB, RS—BHB, S—BHB form, or a mixture thereof.

In some embodiments, the prepared BHB acid and/or salt has a chemical purity of at least 90%.

In some embodiments, the prepared BHB acid and/or salt has a chemical purity of at least 95%, an optical purity of at least about 95%, and/or is formed with a molar yield of at least about 80%.

In some further embodiments, the prepared BHB acid and/or salt has a chemical purity of at least about 98%, an optical purity of at least about 98%, and/or is formed with a molar yield of at least 85%.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

As used herein, the term “about” modifying any amount refers to the variation in that amount encountered in real world conditions of producing chemical products (e.g., acids or salts), such as in the lab, pilot plant, or production facility. For example, an amount of an ingredient employed in a mixture when modified by “about” includes the variation and degree of care typically employed in measuring in BHB acid/salt production plant or lab. Whether or not modified by “about,” the amounts include equivalents to those amounts. Any quantity stated herein and modified by “about” can also be employed in the present invention as the amount not modified by “about.”

As used herein, the term “acid” refers to a substance that is capable of donating a proton (i.e., a hydrogen cation) to form a conjugate base of the acid. Examples of acids include, but are not limited to, acetic acid, formic acid, chloroacetic acid, bromoacetic acid, trichloroacetic acid, and benzoic acid. As used herein, the term “acid” includes, among otherthings, a fatty acid having the chemical formula R—COOH, wherein R is substituted or unsubstituted hydrocarbyl group. A preferred embodiment includes ethyl ester.

As used herein, the term “biobased” means that a substance is created or made in whole or in part with a naturally occurring or existing, or biological, material, rather than a fully man-made or synthesized material.

As used herein, the term “salt” or “salts” refer to salts prepared from bases or acids (e.g., pharmaceutically acceptable non-toxic bases or acids) including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Exemplary preferred embodiments include the ammonium, calcium, magnesium, potassium, and sodium salts. Salts in the solid form may exist in more than one crystal structure, and may also be in the form of hydrates. Salts derived from organic bases include salts of primary, secondary, and tertiary amines, substituted amines including naturally occurring substituted amines, cyclic amines, and basic ion exchange resins, such as arginine, betaine, caffeine, choline, N, N′-dibenzylethylene-diamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. Acids may include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Exemplary embodiments include the citric, hydrobromic, hydrochloric, maleic, phosphoric, sulfuric, fumaric, and tartaric acids.

As used herein, term “administer” “administered” or “administering” means either directly administering a compound or composition of the present invention, or administering a prodrug, derivative or analog which will form an equivalent amount of the active compound or substance within the body.

### DETAILED DESCRIPTION

Reference will now be made in detail to the preferred embodiments of the invention, examples of which are further illustrated. While the invention will be described in conjunction with the preferred embodiments, it will be understood that they are not intended to limit the invention to these embodiments. On the contrary, the invention is intended to cover alternatives, modifications, and equivalents, which may be included within the spirit and scope of the invention as defined by the claims. Furthermore, in the detailed description of the present invention, numerous specific details are set forth in order to provide a thorough understanding of the present invention. However, it will be obvious to one of ordinary skill in the art that the present invention may be practiced without these specific details. In other instances, well-known methods, procedures, components, and other features have not been described in detail as not to unnecessarily obscure aspects of the present invention.

Generally speaking, various embodiments of the present invention provide for synthesis and preparation method of beta-hydroxybutyrate, particularly (R)-beta-hydroxybutyrate (R—BHB), acid and/or salts with pure biobased carbon content via chemical synthesis and/or biosynthesis process, which may include, e.g., biological fermentation or bio-enzymatic method. The processes are simple and environmentally friendly at a low cost and high yield. In particular, the raw materials are critical for successfully synthesizing R—BHB acid and/or salts with pure biobased carbon content.

Accordingly, the BHB (e.g., R—BHB) acids and/or salts according to the present invention contain pure biobased carbon content, thereby achieving safer and healthier R—BHB acid and/or salt products that can be used widely and safely as a dietary supplement, food additives, etc. For instance, the biobased carbon is pure (e.g., 100% as measured by Carbon-14 analysis). In addition, the synthetic BHB (e.g., R—BHB) acid and/or salts according to the present invention have high yield (e.g., a molar yield of 80-100%, preferably 85-97%), and high purity (e.g., chirality of at least 95%, preferably 97%; chemical purity of at least 95%, preferably 98%; and optical purity of 95-100%, preferably 98-100%). Furthermore, the synthesis processes according to the present invention are simple, easy to control and repeat, effective and efficient, and at a low cost.

The following examples are illustrative of select embodiments of the present invention and are not meant to limit the scope of the invention.

### Example 1. Preparation of (R)-3-Hydroxybutanoic Acid Sodium Salt, C₄H₇O₃Na

The preparation method of an exemplary R—BHB salt (i.e., (R)-3-hydroxybutanoic acid sodium salt, C₄H₇O₃Na) includes the following steps:

1. Pump in 2100 kg of purified water into the 5000 L clean and checked reactor, add 220 kg of sodium hydroxide and then stir it. At the same time, pump in 1400 kg of purified water and then add 700 kg of ethyl (R)-3-hydroxybutyrate into the 2000 L configuration kettle of reactor. Stir the mixture well and then set aside.

2. Dropwise to the aqueous solution of ethyl (R)-3-hydroxybutyrate into the aqueous sodium hydroxide solution for 1-2 h at below 20° C.

3. Keep this reactor for 1-2 h at 50-60° C.

4. Sample inspection by IPC-1 middle control analysis. Here are the detailed steps in this process: Dilute 1 mL of the mixture in 10 mL of water and detect by thin-Layer chromatography (TLC) and HPLC.

5. After determining qualified, transfer to the distillation kettle, vacuum distill and dehydrate at 50-60° C. under negative pressure, meanwhile, sample directly and check the content by IPC-2 middle control analysis. After determining the qualified, concentrate to 30% and pack it directly into a barrel, label it, calculate the weight, and transfer it.

6. At last, obtain a white solid (R)-3-hydroxybutanoic acid sodium salt, of which the bio-based carbon content was 100% (ASTM D6866/ISO 16620-2:2015), the molar yield was 92.0%-97.0% and the optical purity was ≥98%.

### Example 2. Preparation of (R)-3-Hydroxybutanoic Acid Calcium Salt and Magnesium Salt (CsH₁₄CaO₆ & C₈H₁₄MgO₆)

The preparation method of an exemplary R—BHB salt (i.e., (R)-3-hydroxybutanoic acid calcium salt and magnesium salt, CsH₁₄CaO₆ and CsH₁₄MgO₆, includes the following steps:

1. Pump in 1500 kg of purified water and add 500 kg of methyl 3-hydroxybutyrate into the clean and checked reactor, and then stir it. At the same time, add 39.9 kg of calcium oxide and 58.7 kg of magnesium oxide.

2. Keep this reactor for 8-10 h at 90-95° C.

3. Sample inspection by IPC-1 middle control analysis. Here are the detailed steps in this process: Dilute 1 mL of the mixture in 10 mL of water and detect by thin-Layer chromatography (TLC) and HPLC.

4. After determining qualified, concentrate under reduced pressure of less than 5 KPa at 65-70° C., and steam out about 1000 kg of water.

5. Reduce the temperature of reactor to 30-40° C., and then discharge and filter solution to remove mechanical impurities, etc.

6. Spray-dry it to obtain the product. And then sample directly and check the content by IPC-2 middle control analysis. After qualified, proceed to the next step such as granulation, sieving, packaging, and other operations according to customer requirements.

7. At last, obtain a white solid (R)-3-hydroxybutanoic acid calcium and magnesium salt, of which the bio-based carbon content was 100% (ASTM D6866/ISO 16620-2:2015), the molar yield was 85.0%-90.0%, total weight yield was 85.0%-90.0% and the chirality was >97%.

### Example 3. Preparation of (R)-Beta-Hydroxybutanoic Acid, C₄H₈O₃

The preparation method of an exemplary R—BHB acid (i.e., (R)-beta-hydroxybutanoic acid, C₄H₈O₃) includes the following steps:

1. Pump in 3000 kg of purified water and add 1000 kg of methyl 3-hydroxybutyrate, into the clean and checked reactor, and then stir it. At the same time, add 250 kg of calcium oxide.

2. Keep this reactor for 6-8 h at 90-95° C.

3. Sample inspection by IPC-1 middle control analysis. Here are the detailed steps in this process: Dilute 1 mL of the mixture in 10 mL of water and detect by thin-Layer chromatography (TLC) and HPLC.

4. After determining qualified, reduce the temperature of reactor to 0-5° C., and extract 424 kg of concentrated sulfuric acid to the upper tank at the same time.

5. Add concentrated sulfuric acid dropwise at 0-5° C., after the dropwise addition, and then stir for 1 h. Discharge and filter (the specific filter equipment and operation are subject to the site). Then soak the filter residue in 200 kg of water, and then drain and filter mixture solution. And then sample mixture solution directly and check the content by IPC-2 middle control analysis. After qualified, transfer mother liquor to distillation kettle.

6. Add 40 kg potassium hydroxide, steam out of water, the amount of water according to the IPC-5 results, vacuum distill and dehydrate mother liquor at 50-60° C. under negative pressure, meanwhile, sample directly and check the content by IPC-3 middle control analysis. After determining the qualified, filter out a small amount of salt and mechanical impurities, obtain mother liquor as the finished product and pack it directly into a barrel, label it, sample and inspect, calculate the weight.

7. At last, obtain a colorless to yellowish liquid (50% aqueous solution) (R)-beta-hydroxybutanoic acid, of which the bio-based carbon content was 100% (ASTM D6866/ISO 16620-2:2015), the molar yield was 90.0%-95.0% and the optical purity was ≥98%.

### Example 4. Biological Fermentation Process of (R)-Beta-Hydroxybutanoic Acid

An exemplary preparation method of R—BHB acid via biological fermentation process includes the following steps.

1. Construction of recombinant strains:

Synthesize the phbA, phbB (from Ralstonia eutropha) and yciA (from E. coli AF1000) genes through optimization of the entire sequence, and then construct them in the vector pRSFDuet-1, and then introduce the recombinant vector into E. coli BL21 (DE3), at last, the E. coli gene Engineering bacteria pRSF-phbA-phbB-yciA/BL21(DE3) was conducted.

2. Fermentation of recombinant bacteria:

Thaw the glycerol tube recombinant strain stored at minus 80° C. and inoculate it into a 500 mL Erlenmeyer flask containing 75 mL seed culture medium (10 g/L sodium chloride, 10 g/L peptone, 5 g/L yeast powder), and cultivate it at 37° C. overnight; and

Inoculate 75 mL of the seed culture into a 5-liter fermenter with 3-liter fermentation medium. The composition of the fermentation medium is 15 g/L glucose, 10 g/L peptone, 5 g/L yeast powder. After sterilization, control the pH value at 6.9-7.0, the fermentation temperature at 30° C., the tank pressure at 0.05 Mpa, the initial aeration ratio of 1 vvm, and the initial stirring speed of 200 rpm. During the fermentation process, control the pH near 7.0 by fed-batching ammonia water and the dissolved oxygen constant (dO2) at 20-30% by adjusting the ventilation and stirring speed. When the dissolved oxygen constant increases rapidly, control the dissolved oxygen at 20-30% by fed-batching glucose continuously. When the OD of cell culture reaches 20, add IPTG to the final concentration of 0.1 mM for induction. After fermentation cultivating for 24 h, obtain 15.2 g/L of (R)-3-hydroxybutyric acid.

3. Extraction and Purification of (R)-Beta-Hydroxybutanoic Acid

Centrifuge the obtained 3-liter fermentation broth at 4500 rpm, and discard the bacterial cells. Filter the supernatant through a ceramic membrane to obtain a clear filtrate, and filter it though a nanofiltration membrane. And then pass the final filter through a 732-cation exchange resin to obtain a concentrated filtrate. Collect the oily concentrated filtrate while it was hot and obtain (R)-beta-hydroxybutanoic acid, of which the bio-based carbon content was 100% (ASTM D6866/ISO 16620-2:2015), the purity was 98% and an optical purity was 100%.

### Example 5 Bio-Enzyme Catalysis of (R)-Ethyl-3-Hydroxybutyrate (BHB Ethyl Ester)

An exemplary preparation method of R—BHB acid via bio-enzyme catalysis includes the following steps.

1. Construction of recombinant strains:

Synthesize SmADH (from Stenotrophomonas maltophilia) gene through optimization of the whole sequence, and construct it in the vector pET32a, and then introduce the recombinant vector into E. coli BL21 (DE3), at last, the E. coli genetic engineering strain pET-ADH/BL21 (DE3) was conducted.

2. Fermentation of recombinant bacteria:

Thaw the glycerol tube recombinant strain stored at minus 80° C. and inoculate it into a 500 mL Erlenmeyer flask containing 75 mL seed culture medium (10 g/L sodium chloride, 10 g/L peptone, 5 g/L yeast powder), and cultivate it at 37° C. overnight; and

Inoculate 75 mL of the seed culture into a 5-liter fermenter containing 3-liter fermentation medium. The composition of the fermentation medium is 15 g/L glucose, 10 g/L peptone, 5 g/Lyeast powder). After sterilization, control the pH value at 6.9-7.0. the fermentation temperature at 30° C., the tank pressure at 0.05 Mpa, and the initial aeration ratio of 1 vvm, the initial stirring speed of 200 rpm. During the fermentation process, control the pH near 7.0 by fed-batching ammonia water and the dissolved oxygen constant (dO2) at 20-30% by adjusting the ventilation and stirring speed. When the dissolved oxygen constant increases rapidly, control the dissolved oxygen at 20-30% by fed-batching glucose continuously. When OD of the cell culture reaches 20, add IPTG to the final concentration of 0.1 mM for induction, and stop the fermentation after inducting for 10 h.

3. Bio-enzyme catalysis:

Centrifuge the fermentation broth, wash the bacteria twice with PBS buffer (100 mM, pH=7.0), and prepare 50 g/L bacterial solution. And measure 1 L bacterial solution, add 700 ml ethyl acetoacetate and 1 L isopropanol, and then catalyze at 30° C., 200 rpm for 10 h. At last, obtain 259.3 g/L of (R)-ethyl-3-hydroxybutyrate, of which the conversion rate was 98.2%, and the optical purity was 100%.

4. Extraction and purification of (R)-ethyl-3-hydroxybutyrate:

Centrifuge the final solution after enzyme-catalyzed reaction, discard the precipitate, and obtain the supernatant. Rectify the supernatant and then collect the (R)-ethyl-3-hydroxybutyrate component. At last, obtain the (R)-ethyl-3-hydroxybutyrate with the bio-based carbon content of 100% (ASTM D6866/ISO 16620-2:2015) and the purity of 99.5%.

The prepared BHB acid or salts with pure biobased carbon content are healthier and safer than the conventional R—BHB acid and/or salts in the market. Postbiotics are byproducts of probiotic bacterial (e.g., E. coli) fermentation. Common postbiotics include short-chain fatty acids, such as butyrate. Under the biological technology (e.g., Biological fermentation, and Bio-enzyme catalysis), the inventors of the present invention analyzed butyrate content of the products ((R)-beta-hydroxybutanoic acid, BHB ethyl ester) by HPLC, and found all these products include butyrate. The products ((R)-beta-hydroxybutanoic acid, BHB ethyl ester) obtained by the biological technology contain more nutrients such as postbiotics. The postbiotics play an important and beneficial role in the health of the body, such as helping intestinal health, improving immunity, improving gastric function, etc. As such, the BHB (e.g., R—BHB) acid and/or salts with pure biobased carbon content according to the present invention may be widely used in dietary supplementary, food additives and/or pharmaceuticals, and are advantageous for human body and environment.

Although specific embodiments and examples of this invention have been illustrated herein, it will be appreciated by those skilled in the art that any modifications and variations can be made without departing from the spirit of the invention. The examples and illustrations above are not intended to limit the scope of this invention. Any combination of embodiments of this invention, along with any obvious their extension or analogs, are within the scope of this invention. Further, it is intended that this invention encompass any arrangement, which is calculated to achieve that same purpose, and all such variations and modifications as fall within the scope of the appended claims.

All the features disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example of a generic series of equivalent or similar features.

### OTHER EMBODIMENTS

It is to be understood that while the invention has been described in conjunction with the detailed description thereof and accompanying figures, the foregoing description and accompanying figures are only intended to illustrate, and not limit the scope of the invention, which is defined by the scope of the appended claims. Other aspects, advantages, and modifications are within the scope of the following claims. All publications referenced herein are incorporated by reference in their entireties.

What is claimed is:

1. A composition comprising a beta-hydroxybutyrate (BHB) acid or salt, wherein the BHB acid or salt comprises biobased carbon content, and the percentage of biobased carbon content is about 100% as measured by Carbon-14 analysis.

2. The composition of claim 1, wherein the BHB acid further comprises an ethyl alcohol which reacts with the BHB acid to form a BHB ethyl ester.

3. The composition of claim 1, wherein the BHB salt comprises a BHB metal salt.

4. The composition of claim 3, wherein the BHB salt is formed from sodium, calcium, magnesium, or a mixture thereof.

5. The composition of claim 1, wherein the BHB salt comprises R—BHB, RS—BHB, S—BHB form, or a mixture thereof.

6. The composition of claim 1, wherein the BHB acid or salt has a chemical purity of at least about 95%, an optical purity of at least about 95%, or is formed with a molar yield of at least about 80%.

7. The composition of claim 1, wherein the composition is administrated to a mammal for promoting or sustaining ketosis in the mammal.

8. A beta-hydroxybutyrate (BHB) acid or salt comprising about 100% biobased carbon content.

9. The beta-hydroxybutyrate (BHB) acid of claim 8, wherein the BHB acid further comprises an ethyl alcohol which reacts with the BHB acid to form a BHB ethyl ester.

10. The beta-hydroxybutyrate (BHB) salt of claim 8, wherein the BHB salt is formed with a metal ion.

11. A method for preparing beta-hydroxybutyrate (BHB) acid or salt with pure biobased carbon content, comprising a chemical synthesis or biosynthesis process for enabling pure biobased carbon content in the prepared R—BHB acid or salts, wherein the chemical synthesis or biosynthesis processes comprises a biological fermentation or a bio-enzymatic method.

12. The method of claim 11, wherein the method comprises a biosynthesis process.

13. The method of claim 12, wherein the method comprises steps of (a) construction of recombinant strains; (b) fermentation of the recombinant bacteria; and (c) extraction and purification of BHB acid or salt.

14. The method of claim 13, further comprising a step of bio-enzyme catalysis after the process of fermentation of the recombinant bacteria.

15. The method of claim 11, wherein the percentage of biobased carbon content is about 100% as measured by Carbon-14 analysis.

16. The method of claim 11, wherein the prepared BHB acid comprises a BHB ethyl ester.

17. The method of claim 11, wherein the prepared BHB salt comprises a BHB metal salt.

18. The method of claim 17, wherein the prepared BHB salt is formed from sodium, calcium, magnesium, or a mixture thereof.

19. The method of claim 11, wherein the prepared BHB salt comprises R—BHB, RS—BHB, S—BHB form, or a mixture thereof.

20. The method of claim 11, wherein the prepared BHB acid or salt has a chemical purity of at least about 95%, an optical purity of at least about 95%, or is formed with a molar yield of at least about 80%.
